(19) United States
(12) Patent Application Publication
(10) Pub. No.: US 2003/0228572 A1
(43) Pub. Date: Dec. 11, 2003

(54) Method of determining the efficacy of a compound in treating microbial infection

(76) Inventors: Tsung C. Chang, Tainan (TW); Jiunn Jong Wu, Tainan (TW); Ay Huei Huang, Tainan (TW); Jin Hwa Dai, Hsinchu (TW); Jen Jih Tsen, Da Sor (TW)

Correspondence Address: FISH & RICHARDSON PC, 225 FRANKLIN ST, BOSTON, MA, 02110, US

(21) Appl. No.: 10/412,619

(22) Filed: Apr. 11, 2003

Related U.S. Application Data
(63) Continuation-in-part of application No. 09/073,155, filed on May 5, 1998, now abandoned.

(51) Int. Cl. C12Q 1/70; C12Q 1/18

(52) U.S. Cl. 435/5; 435/32

(57) ABSTRACT

A method of determining the efficacy of a compound in treating a subject infected with a microbe, or a method of determining the minimum inhibitory concentration of an antimicrobial agent against a microbe. The method involves the measurement of an electrical parameter of a culture of the microbe.

### FIELD OF THE INVENTION

This invention relates to the use of electrochemical techniques to examine growth of microbes in the presence of antibiotics.

### BACKGROUND OF THE INVENTION

Approximately 200,000 cases of septicemia occur annually in the United States, with a high rate of mortality (Washington et al., J. Appl. Bacteriol., 67:575-588, 1986). The incidence rates of bacteremia and fungemia were reported to be 3.4 to 28 per 1,000 hospital admissions and were estimated to average 10 per 1,000 admissions (1%) in the United States (Weinstein et al., Rev. Infect. Dis., 5:35-53, 1983). The five most common isolates from blood cultures were Escherichia coli, Staphylococcus aureus, Klebsiella pneumoniae, Pseudomonas aeruginosa, and Streptococcus pneumoniae (Brauner et al., Acta Pathol. Microbiol. Immunol. Scand., 99:381-386, 1991). Positive blood cultures are normally streaked on appropriate media, followed by species identification and antibiotic susceptibility testing. The whole procedures may take as long as 2 days.

### SUMMARY OF THE INVENTION

The invention features a method of determining the efficacy of a known compound in treating a patient or an animal infected (i.e., known to be, or suspected of being, infected) with a microbe. In this method, a bodily sample known to contain or suspected of containing the microbe is obtained from a subject and added to a medium (liquid or solid) containing the known compound; and while the medium is being incubated under conditions that allow the microbe to grow, an electrical parameter (e.g., impedance, conductance, or capacitance) of the culture medium is measured over a pre-determined period of time, where the absence of an accelerating change of the electrical parameter within this period of time indicates that the compound is effective in treating the subject's infection. The microbe can be a bacterium (e.g., Escherichia coli, Staphylococcus aureus, Klebsiella pneumoniae, Pseudomonas aeruginosa, or Streptococcus pneumoniae), a fungus such as yeast, or a protozoa. The electrical parameter to be measured can be e.g., conductance, impedance, or capacitance. The featured methods can be used to determine whether a subject infected with a bacterium (e.g., Staphylococcus aureus) can be treated with any given antibiotic (e.g., oxacillin or methicillin). An antibiotic is a compound that is capable of inhibiting the growth of a microbe such as a bacterium or a fungus.

Also embraced by the invention is a method of measuring the minimum inhibitory concentration (“MIC”) of an antibiotic against a microbe. According to the method, an identical amount of a microbe-containing sample (e.g., a bodily sample) is first added to a plurality (e.g., more than 3) of culture media, the culture media being identical except that each of them contains a different concentration (e.g., at a serial dilution such as two-fold dilution) of the antibiotic. The culture media are then incubated under conditions that allow the microbe to grow, and during the incubation, an accelerating change of an electrical parameter is detected in each medium within a predetermined period of time, where the lowest antibiotic concentration at which the change is not detected within the period is defined as the MIC.

Other features and advantages of the invention will be apparent from the following detailed description, and from the claims.

### BRIEF DESCRIPTION OF THE DRAWINGS

FIG. 1 is a graph showing the impedance curves of oxacillin sensitive S. aureus ATCC 29213 (OSSA) grown on Mueller-Hinton agar containing 0 (curve a), 0.063 (curve b), 0.125 (curve c), and 0.25 μg (curve d) oxacillin per ml. The DTs for curves a, b, and c were 4.0, 4.1, and 4.1 h, respectively. No DT was obtained for curve d within 16 hours.

FIG. 2 is a graph showing the impedance curves of the oxacillin-resistant S. aureus ATCC 33592 grown on Mueller-Hinton agar containing 0 (curve a), 32 (curve b), 64 (curve c), and 128 μg (curve d) oxacillin per ml. The detection times for curves a, b, c, and d were 5.3, 6.3, 7.0, and 9.3 h, respectively.

FIG. 3 is a graph showing the impedance curves of the oxacillin-resistant S. aureus ATCC 33591 grown on Mueller-Hinton agar containing 0 (curve a), 32 (curve b), 64 (curve c), and 128 μg (curve d) oxacillin per ml. The detection times for curves a, b, c, and d were 5.3, 6.2, 6.8, and 12.1 h, respectively.

### DETAILED DESCRIPTION OF THE INVENTION

One method featured in the invention is used to determine the efficacy of a known compound (e.g., an antibiotic) in treating a subject infected with a microbe. In the method, a bodily sample from the subject is added to a culture medium containing the compound. It may be desirable to adjust the microbe concentration in the bodily sample with a culture medium prior to adding the sample to the compound-containing medium. An electrical parameter of the medium is monitored while the medium is being incubated in the compound-containing medium under conditions that allow the microbe to grow, and the absence of an accelerating change of the parameter within a predetermined period of time indicates that the compound is effective in treating the infection. In conventional methods of determining the efficacy of a drug in treating an infection by a microbe, the microbe needs to be first obtained as a pure isolate (i.e., a colony) by streaking a microbe-containing sample on an agar plate. This method eliminates such a requirement, thus facilitating timely and effective treatment of infected subjects. Further, as shown in the example below, a test performed according to this method has unexpectedly high specificity as well as sensitivity. In other words, the results obtained by the test are in high agreement with those obtained by conventional methods. Thus, the test is not only fast, but also accurate. These advantages are especially desirable in identifying drugs for treating life-threatening infections such as bacteremia.

This invention also features a method of determining the MIC of an antibiotic against a microbe. In this method, an identical amount of a microbe-containing sample is added to a plurality of culture media containing various concentrations of the antibiotic, and the culture media are incubated under conditions that allow the microbe to grow. During the incubation, an accelerating change of an electrical parameter in each of the media is observed within a preset period of time, where the lowest antibiotic concentration at which no DT is detected within the preset period is the MIC. For many clinically important microbial species, the National Committee for Clinical Laboratory Standards (“NCCLS”) list drug breakpoint concentrations that differentiate microbial strains resistant to a given drug from strains sensitive to this drug. For instance, the breakpoint concentrations of cephalothin are 8 and 32 μg/ml for E. coli; that is, if an E. coli strain has a MIC lower than or equal to 8 μg/ml, it is considered sensitive to cephalothin, and if an E. coli strain has a MIC equal to or higher than 32 μg/ml, it is considered resistant. Once the MIC of a given drug is obtained by this method for a strain of a known microbial species, one can readily determine whether this strain is resistant or sensitive to the drug by using the NCCLS.

In both of the two above-described methods, an electrical parameter of a culture medium changes because proliferating microbes breakdown the substrate molecules in the culture medium to smaller molecules (e.g. acids), which have more charges than the substrate itself. As the microbes grow, the impedance of the medium decreases, whereas the conductance increases. When the microbes grow to a population of approximately 107 CFU/ml or higher, an accelerating change of the parameter will occur. The time point when this occurs is termed detection time (“DT”). Thus, the absence of an accelerating change of a selected electrical parameter (i.e., the absence of a DT) within a predetermined period indicates that the growth of the test microbe is slowed down, or even completely inhibited, by the test compound.

An appropriate time period for observing an accelerating change of the electrical parameter can be predetermined empirically. The time period can be as long as the DT of a negative control, i.e., a microbe culture identical to the test culture except that the former does not contain the test compound. However, a longer time period is preferred. For instance, the predetermined time period can also be two, three or four times as long as the DT of a negative control, or it can be conveniently set as, e.g., 16, 20, or 24 hours. The predetermined time period can also be the DT of a negative control plus a few more hours (e.g., about 3 to 5 hours). A predetermined time period varies with the strain of the test microbe, the culture conditions, the concentration of the antimicrobial compound used, etc., but preferably, is sufficiently long to allow differentiation between strains sensitive and resistant to the test compound. Microbial strains known to be resistant or sensitive to the test compound can be used to set the predetermined time period.

A bodily sample used in the present methods can be sputum, throat swabs, blood, urine, cerebrospinal fluid, skin, saliva, synovial fluid, bronchial wash, bronchial lavage, biopsy, or other tissue or fluid samples taken from human patients or veterinary subjects.

Media used to grow the test microbe can be selected based on well known techniques. See, e.g., NCCLS; Woods et al., Manual of Clinical Microbiology, 6th ed., American Society for Microbiology, Washington, D.C. Electrical parameters and DTs of microbe cultures can be measured by methods well known in the art (see, e.g., U.S. Pat. No. 5,591,599). Microbiological analyzers particularly useful for such measurement include, but are certainly not limited to, BACTOMETER M128 (BioMerieux Vitek, Hazelwood, Mo.), MALTHUS 2000 (Malthus Instruments, Britain), and MICROSCAN-W/A (Baxter Diagnostics, West Sacramento, Calif.). The use of these automated analyzers facilitates simultaneous testing of multiple drugs.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can be used in the practice or testing of the present invention. All citations herein are incorporated by reference in their entirety. In case of conflict, the present specification, including definitions, will control. The materials, methods, and examples are illustrative only and not intended to be limiting.

The following example is meant to illustrate the methods of the present invention and the materials used in the methods. Suitable modifications and adaptations of the described conditions and parameters are-within the spirit and scope of the present invention.

Bacterial Strains, Media, and Reagents

Among the 238 stock cultures of S. aureus tested by the impedance method, ATCC 29213 (oxacillin-sensitive S. aureus or “OSSA”), ATCC 33591 (oxacillin-resistant S. aureus or “ORSA”) and ATCC 33592 (ORSA) were obtained from the American Type Culture Collection (“ATCC,” Rockville, Md.). The remaining 235 strains (149 ORSA and 86 OSSA) were isolated at the National Cheng Kung University Hospital (Tainan, Taiwan) from various clinical specimens. Oxacillin susceptibility of the clinical isolates was determined by the standardized disc diffusion method (National Committee for Clinical Laboratory Standards, Performance standards for antimicrobial disk susceptibility tests, 5th ed., Approved standard M2-A5. National Committee for Clinical Laboratory Standards, Villanova, Pa., 1993). Oxacillin was a product from Sigma Chemical Co. (St. Louis, Mo.). Mueller-Hinton agar, tryptic soy agar (“TSA”), tryptic soy broth (“TSB”) and other media were purchased from Difco Laboratories (Detroit, Mich.).

Effect of Oxacillin Concentration on the Impedance Curves

Mueller-Hinton agar containing different concentrations of oxacillin (0 to 128 μg) was prepared by two-fold serial dilutions, and 0.6 ml of the medium was dispensed into the module wells of BACTOMETER M-128 (bioMerieux Vitek, Hazelwood, Mo.). The three ATCC S. aureus strains were used to observe the effect of oxacillin concentration on the impedance curves during bacterial growth. These strains were subcultured on TSA for 18 to 24 hours (“h”) at 37° C. One single colony on TSA was inoculated into 10 ml of TSB, incubated at 37° C. for several hours, and diluted with 0.1% peptone water to a turbidity of 0.5 McFarland. An aliquot (0.1 ml) of the bacterial suspensions was inoculated into the module well containing Mueller-Hinton agar supplemented with oxacillin. The inoculated modules (each module contains 16 wells) were inserted into the incubator of BACTOMETER set at 37° C. The impedance change in each well was continuously monitored and recorded by the instrument at 6-minute (“min”) intervals for 24 h, and results were obtained graphically as impedance growth curves. DT in hours for each well was automatically determined by the instrument software when three consecutive readings of impedance change exceeded the default value in the instrument, or was manually determined by locating the inflection point (where an accelerating change of impedance was evident) on the impedance curve.

Oxacillin Susceptibility Tests of Clinical S. aureus Isolates by the Impedance Method

Susceptibility tests of the 235 clinical isolates of S. aureus were performed in a similar way to that used for the ATCC strains, except that only one concentration (2 μg/ml) of oxacillin in Mueller-Hinton agar was used. A negative control (Mueller-Hinton agar without oxacillin) was included for each strain tested. A strain was designated as OSSA if, in the presence of the antibiotic, there was no DT obtained within an incubation period of 24 h. A strain was designated as ORSA if, in the presence of the antibiotic, the DT was not affected or the delay in DT was less than 3 h as compared to the negative control. S. aureus ATCC 29213 (OSSA) and ATCC 33592 (ORSA) were run at the same time as controls. Strains showing discrepant results were reconfirmed by the oxacillin screen agar (Becton Dickinson Microbiology Systems, Cockysville, Md.).

Detection of ORSA in Blood Cultures by the Impedance Method

Blood specimens were collected at the National Cheng Kung University Hospital during a period in 1996. The BACTEC NR6A and NR7A vials (containing about 30 ml of liquid media; Becton Dickinson Microbiology Systems) were normally inoculated with 3 to 5 ml of blood from the patients, inserted into a BACTEC NR660 instrument (Becton Dickinson Microbiology Systems), and incubated at 37° C. A small aliquot (0.1 to 0.2 ml) of the culture broth was drawn from the vials showing growth of gram-positive cocci (as determined by Gram stain), heated in a boiling water bath for 15 min, and tested for thermonuclease activities on slides coated with toluidine blue-DNA agar (Bennett et al., Staphylococcus aureus, pp. 161-166, In: Bacteriological Analytical Manual, 7th ed. Association of Official Analytical Chemists International, Arlington, Va., 1992). A positive thermonuclease reaction was the development of a pink halo extending at least 1 mm from the periphery of the reaction well. An aliquot (0.1 ml) of the positive blood cultures demonstrating thermonuclease activities was inoculated into the BACTOMETER module wells containing Mueller-Hinton agar (the negative control) or the same medium supplemented with oxacillin (2 μg/ml). The modules were incubated at 37° C. and DTs of the wells were automatically determined by the instrument or by manual inspection of the impedance curves. A vial was designated as ORSA or OSSA positive according to the same parameters set for pure stock cultures. The presence of S. aureus in the blood culture vials was confirmed by the conventional culture and identification methods, while oxacillin susceptibility of the isolates was analyzed by the agar disc diffusion method (National Committee for Clinical Laboratory Standards, Performance standards for antimicrobial disk susceptibility tests, 5th ed., Approved standard M2-A5. National Committee for Clinical Laboratory Standards, Vilanova, Pa., 1993). Positive blood culture vials showing growth of mixed cultures, as revealed by Gram stain, were not included in this study.

Sensitivity and specificity

Sensitivity and specificity were determined as described by McClure (McClure, F. D., J. Assoc. Off. Anal. Chem., 73:953-960, 1990).

Results

(1) Effect of Oxacillin Concentration on Impedance Curves

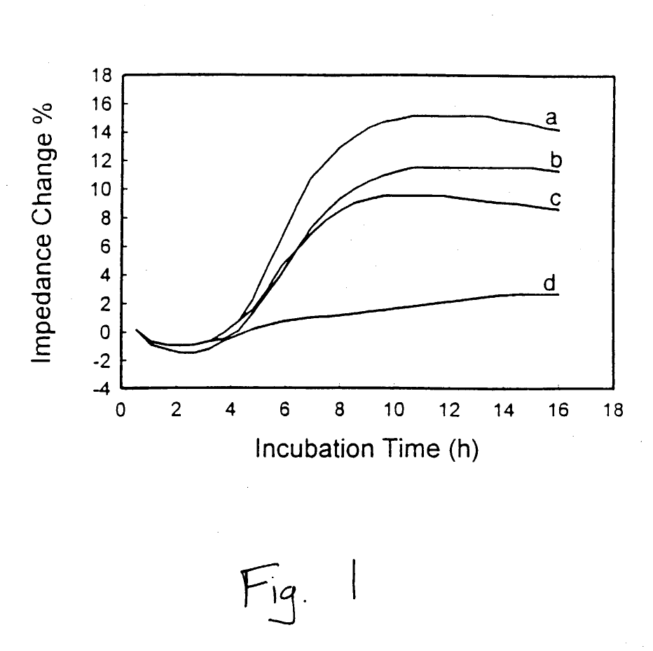

The impedance curves of S. aureus ATCC 29213 (OSSA) grown under different concentrations (0 to 0.25 μg/ml) of oxacillin are shown in FIG. 1. In the absence of oxacillin, the test strain had a DT of 4 h (FIG. 1, curve a), and there was a small increase (0.1 h) in DTs at oxacillin concentrations of 0.063 and 0.125 μg/ml (FIG. 1, curves b and c, respectively). However, when the oxacillin concentration increased to 0.25 μg/ml, the growth of S. aureus ATCC 29213 was almost completely inhibited and no DT was obtained (FIG. 1, curve d). It was noted that a large decrease in the final values of impedance change occurred for the three impedance curves (FIG. 1, curves b, c, and d) in the presence of oxacillin.

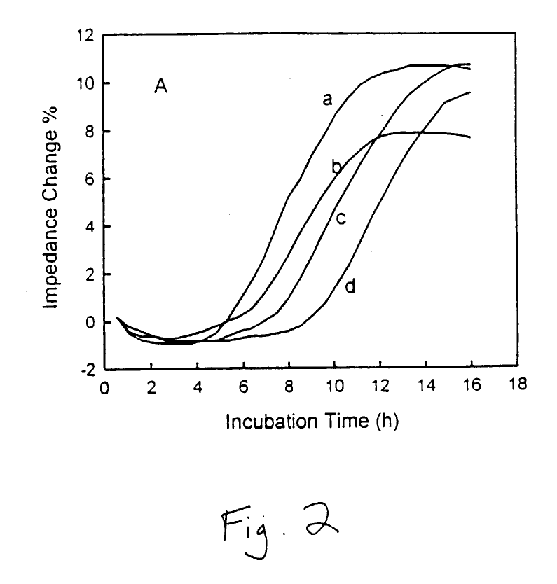

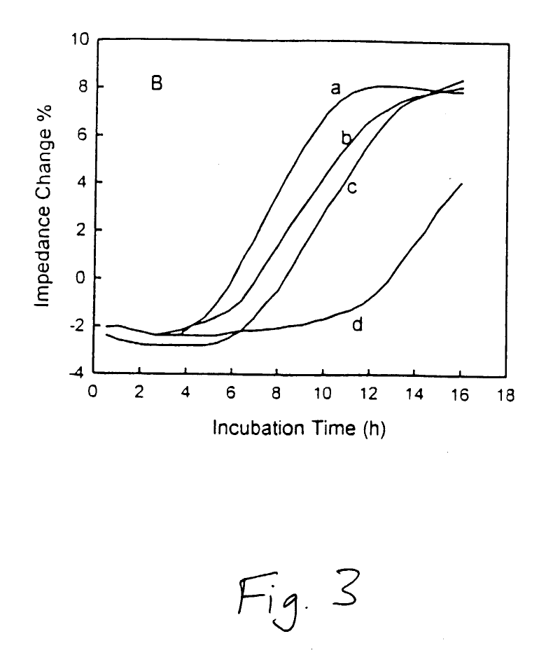

In contrast, the growth of S. aureus ATCC 33592 (FIG. 2) and 33591 (FIG. 3), both of which are oxacillin-resistant, was only slightly inhibited at an oxacillin concentration as high as 64 yg/ml (FIGS. 2A and 2B, curves c). For ATCC 33592, the DTs increased from 5.3 h in the absence of the antibiotic to 6.3, 7.0, and 9.3 h, respectively, in the presence of 32, 64, and 128 μg of oxacillin per ml of the test medium. There was a similar trend in the delay of DTs of ATCC 33591 in the presence of the antibiotic (FIG. 3). It seemed that S. aureus ATCC 33592 was more resistant than ATCC 33591 at high concentration of oxacillin. At an oxacillin concentration of 128 μg/ml, ATCC 33592 (FIG. 2, curve d) still had a typical impedance curve having a high value of impedance change and a drastic change in the slope of the impedance curve at the DT point. However, at the same concentration (128 μg/ml) of oxacillin, ATCC 33591 displayed an impedance curve with a relatively lower value of impedance change and had a DT of 12.1 h (FIG. 3, curve d).

The growth of the two oxacillin-resistant strains (ATCC 33592 and 33591) was not inhibited to a large extent even at an oxacillin concentration as high as 128 μg/ml. This was in strong contrast with the oxacillin-sensitive strain (ATCC 29213) whose growth was almost completely inhibited at an oxacillin concentration of 0.25 μg/ml. Thus, ORSA and OSSA strains can be distinguished by the impedance method.

(2) Susceptibility Tests of Clinical S. aureus Isolates by the Impedance Method

Since an S. aureus strain for which the MIC was s2 μg was defined as oxacillin-sensitive by the MIC method described by National Committee for Clinical Laboratory Standards, Methods for Dilution Antimicrobial Susceptibility Tests for Bacteria that Grow Aerobically, 3rd ed., Approved standard M7-A3, National Committee for Clinical Laboratory Standards, Villanova, Pa., 1993; Woods et al., Manual of clinical microbiology, 6th ed., American Society for Microbiology, Washington, D.C., 1995, this concentration was used in the impedance method to screen a battery of 235 clinical isolates of S. aureus (86 OSSA and 149 ORSA).

Of the 86 OSSA strains tested, 84 had no DT within an incubation period of 24 h and were considered oxacillin-sensitive by the impedimetric technique (Table 1). Two OSSA strains (strain no. 64 and 73) had DTs similar or equal to the DTs of the negative controls (no oxacillin in the Mueller-Hinton agar) and were recognized as ORSA by the impedance method. The resistance of the two strains to oxacillin were reconfirmed by using the oxacillin screen agar. The 84 strains of OSSA had an average DT of 4.5+0.4 h (range, 4.1 to 6.0 h) (Table 1) when grown in the absence of oxacillin. Of the 86 OSSA strains tested, the specificity of the impedance method was 97.7% (84 of 86).

Among the 149 strains of ORSA tested, 141 had DTs equal to the DTs of the negative controls or the delay in DTs was less than 3 h and were considered oxacillin resistant by the impedimetric technique (Table 1). Among the eight false-negative strains (strain no. 43, 69, 127, 161, 169, 173, 177,and 181), seven had no DT within an incubation period of 24 h, with the remaining one (no. 127) having a DT of 22.7 h that was 17 h behind the negative control (DT=5.7 h). The oxacillin susceptibility of these eight strains was confirmed by using the oxacillin screen agar. The 141 ORSA strains had an average DT of 5.7±0.6 h (range: 4.4 to 6.7 h) (Table 1) in the absence of oxacillin.

TABLE 1
Results of oxacillin susceptibility test and the
DT's of clinical S. aureus isolates obtained by the
impedance method.

| | | | DT in h (range) | |
|---|---|---|---|---|
| | No. of strains | | on Mueller-Hinton agar^{a} | |
| | | | | With |
| Bac- | | Correctly | Without | Oxacillin |
| terium | Tested | Identified | Oxacillin | (2 μg/ml) |
| OSSA | 86 | 84 | 4.5 ± 0.4 (4.1-6.0)^{b} | —^{c} |
| ORSA | 149 | 141 | 5.7 ± 0.6 (4.4-6.7) | 6.0 ± 0.7 (4.7-9.1) |

^{a}Values are means ± standard deviations
^{b}Significant difference from the other value in the column by the unpaired t test at p < 0.0001.
^{c}—, DT was not found within an incubation period of 24 h.

The average DT increased to 6.0±0.7 h (range: 4.7 to 9.1 h) in the presence of oxacillin (2 μg/ml). After 149 ORSA strains had been tested, a sensitivity of 94.6% (141 of 149) was obtained by the impedance method. The overall agreement of the method with the conventional disc diffusion method was (84+141)/(86+149), i.e., 95.7%.

Notably, the average DT (5.7 h) of the ORSA strains in the absence of the antibiotic was significantly (p<0.0001) higher than the average DT (4.5 h) of the OSSA strains, as determined by the unpaired t test (Table 1). This indicates that the growth of most strains of OSSA was faster than ORSA in Mueller-Hinton agar.

(3) Direct Detection of ORSA in Blood Cultures

The blood culture vials showing growth of gram-positive cocci and displaying thermonuclease activities indicated that there were S. aureus in these vials (Madison et al., J. Clin. Microbiol., 18:722-724, 1983). A total of 96 such blood cultures were used for direct detection of ORSA by the impedance method. Among the 38 vials containing ORSA as determined by the conventional methods, 36 displayed typical impedance curves of ORSA with an average DT of 5.5 h. Among the 58 blood cultures containing OSSA, 57 exhibited typical impedance curves having no DTs within an incubation period of 24 h. Therefore, the impedance method had a sensitivity of 94.7% (36 of 38) and a specificity of 98.3% (57 of 58) for the detection of ORSA in blood cultures, and had an agreement of 96.9% (i.e., (36+57)/(38+58)) with the conventional culture techniques comprising strain isolation and the subsequent antibiotic susceptibility tests.

### Other Embodiments

It is to be understood that while the invention has been described in conjunction with the detailed description thereof, that the foregoing description is intended to illustrate and not limit the scope of the invention, which is defined by the scope of the appended claims.

Other aspects, advantages, and modifications are within the scope of the following claims.

What is claimed is:

1. A method of determining the efficacy of a known compound in treating a subject infected with a microbe, the method comprising:
obtaining a bodily sample containing the microbe from the subject;
adding the sample to a medium containing the compound;
incubating the medium under conditions that allow the microbe to grow; and
measuring an electrical parameter of the culture 1I medium over a pre-determined period of time; wherein the absence of an accelerating change of the electrical parameter within the period of time indicates that the compound is effective in treating the subject.

2. The method of claim 1, wherein the electrical parameter is conductance.

3. The method of claim 2, wherein the bodily sample is a blood sample.

4. The method of claim 3, wherein the microbe is Staphylococcus aureus, and the compound is oxacillin or methicillin.

5. The method of claim 1, wherein the microbe is a bacterium.

6. The method of claim 5, wherein the electrical parameter is conductance.

7. The method of claim 6, wherein the bodily sample is a blood sample.

8. The method of claim 7, wherein the microbe is Staphylococcus aureus, and the compound is oxacillin or methicillin.

9. The method of claim 5, wherein the bodily sample is a blood sample.

10. The method of claim 9, wherein the microbe is Staphylococcus aureus, and the compound is oxacillin or methicillin.

11. A method of measuring the minimum inhibitory concentration of an antimicrobial agent against a microbe, the method comprising:
adding an identical amount of a sample containing the microbe to a plurality of culture media, the culture media being identical except that each of them contains a different concentration of the antimicrobial agent;
incubating the plurality of culture media under conditions that allow the microbe to grow; and
in each of the culture media, detecting an accelerating change of an electrical parameter within a predetermined period of time to identify the lowest concentration of the antimicrobial agent at which the change is not detected within the period of time, wherein said lowest concentration of the antimicrobial agent is the minimum inhibitory concentration.

12. The method of claim 11, wherein the sample is a bodily sample from a subject.

13. The method of claim 11, wherein the electrical parameter is conductance.

14. The method of claim 13, wherein the microbe is a bacterium.

15. The method of claim 12, wherein the electrical parameter is conductance.

16. The method of claim 15, wherein the sample is a blood sample.

17. The method of claim 16, wherein the microbe is Staphylococcus aureus, and the compound is oxacillin or methicillin.

18. The method of claim 12, wherein the microbe is a bacterium.

19. The method of claim 18, wherein the electrical parameter is conductance.

20. The method of 19, wherein the sample is a blood sample.

21. The method of claim 20, wherein the microbe is Staphylococcus aureus, and the compound is oxacillin or methicillin.

22. The method of claim 18, wherein the sample is a blood sample.

23. The method of claim 22, wherein the microbe is Staphylococcus aureus, and the compound is oxacillin or methicillin.
